(19) United States
(12) Patent Application Publication
(10) Pub. No.: US 2011/0107491 A1
(43) Pub. Date: May 12, 2011

(54) Welding Helmet Light/Strike Light

(76) Inventors: Paul David Sanders, Boring, OR (US); Christopher Eric Wills, Portland, OR (US)

(21) Appl. No.: 12/902,153

(22) Filed: Oct. 12, 2010

Related U.S. Application Data
(60) Provisional application No. 61/255,961, filed on Oct. 29, 2009.

(51) Int. Cl. A61F 9/06

(52) U.S. Cl. 2/8.2

(57) ABSTRACT

A light assembly that can be attached to virtually any welding helmet to create a well lit working area. The light emitting diode (LED) provides a bright white colored light, which is ideal while doing repair welds, fabrication, and the like. The adjustable light enables the welder to position the light as needed. The product substantially eliminates the need to work in poorly lit conditions by providing welders with the lighting means for starting their welds and inspecting their work, without the need to raise and lower their helmet.

### REFERENCE TO RELATED APPLICATIONS

This patent application claims the benefit of U.S. Provisional Application No. 61/255,961 filed on Oct. 29, 2009, the disclosure of which is incorporated herein by reference in its entirety.

### BACKGROUND OF THE INVENTION

1. Field of the Invention

The present invention relates to a light assembly that can be attached to virtually any welding helmet to create a well lit working area. The light emitting diode (LED) provides a bright white colored light, which is ideal while doing repair welds, fabrication, and the like. The adjustable light enables the welder to position the light as needed. The product substantially eliminates the need to work in poorly lit conditions by providing welders with the lighting means for starting their welds and inspecting their work, without the need to raise and lower their helmet.

2. Description of Related Art

Typically, in most welding situations there is a lack of available light. Many times welders have to position themselves under a vehicle, in a passageway of a ship, and other such dimly lit areas to perform the weld. Even seemingly well-lit shops can provide insufficient direct lighting in certain locations. In order to see what they are doing, welders have to continuously raise and lower their helmets during welding to start their welds and inspect their work. This is inconvenient and unnecessary.

To resolve this inconvenience, mounting illumination equipment on or inside the welder helmet for providing lighting is known in the prior art. More specifically, by way of example, U.S. Pat. No. 4,332,004 to Slaughter discloses a lighting system for a welder's helmet which includes a high intensity, high Kelvin temperature electric light source attached to the face shield for directing a higher energy light beam forwardly of the viewing port.

U.S. Pat. No. 6,340,234 B1 to Brown, Jr. discloses a means to illuminate the lens of a face shield to be worn by a welder. The lens is illuminated to enable the welder to see through the lens prior to the welding are being lighted.

U.S. Pat. No. 7,161,116 B2 to Steinemann discloses a welding protective mask, which includes illumination equipment for illuminating a work area. The illumination equipment includes an illumination device, a detection device for detecting an ambient light intensity, energy storage for electrically supplying the illumination equipment and controller for controlling an intensity of light radiated by the illumination means according to the ambient light intensity. In this prior art, the intensity of the radiant light is in dependence of a detected ambient light intensity. The direction of the light source is not adjustable.

Although these illumination equipment in prior art fulfill their respective, particular objective and requirements, the aforementioned patents do not provide adjustable light source. A more efficient method of providing an adjustable light source is needed for illuminating a working area. In this respect, the adjustable light assembly for the welder helmet and the welder helmet with an adjustable light assembly according to the present invention substantially departs from the conventional concepts and design of the prior art.

### SUMMARY OF THE INVENTION

The present invention discloses a light assembly that can be attached to virtually any welding helmet to create a well lit working area. The light emitting diode (LED) provides a bright white colored light, which is ideal while doing repair welds, fabrication, and the like. The adjustable light enables the welder to position the light as needed. The product substantially eliminates the need to work in poorly lit conditions by providing welders with the lighting means for starting their welds and inspecting their work, without the need to raise and lower their helmet.

A primary object of the present invention is to provide a light assembly for welder to mount on the welder helmets. The light assembly can be retrofitted onto existing helmets or manufactured as an integral part of new welder's helmets.

Another object of the invention is to provide a light assembly which can be easily controlled (switched on or off and adjusted intensity) by the person performing the welding task.

A further object of the present invention is to provide an adjustable light assembly to enable the welder to position the light beams in the desired direction as needed.

Still yet another object of the present invention is to provide a light assembly with an adjustment knob on the helmet so that the users have easy access to position the light beam in the desired direction as needed.

More particularly, the present invention is a welder's helmet light that can be attached to virtually any welding helmet to create a well lit working area. The present invention comprises a small LED light substantially contained within a plastic housing. The light itself may be covered with a clear plate. A battery pack may also be included to power the light. The unit may contain an accessible power switch and intensity adjustment knobs. The light may be mounted onto the top and/or face of a welding helmet. It may be mounted in such a way that it adjustably illuminates the area in front of the helmet's viewing window. Adjustment knobs may be included on the housing, which allow the user to position the light beam in the desired direction. The LED lamp may be approximately 2″ in height, 3″ in length, and 2″ in width. The LED lamp is designed to provide a bright white light source. It may be incorporated into the helmet during manufacture. The exact specifications may vary. The present invention can be retrofitted onto existing helmets or manufactured as an integral part of new welder's helmet.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a cross sectional view from the side of the light assembly 1

FIG. 2 is a cross sectional view from the top of the light assembly 1

FIG. 3 is a cross sectional view from the front of the light assembly 1

FIG. 4 is a perspective view showing the light assembly 1 mounted onto the top of a welder helmet 6.

### DESCRIPTION OF THE PREFERRED EMBODIMENT

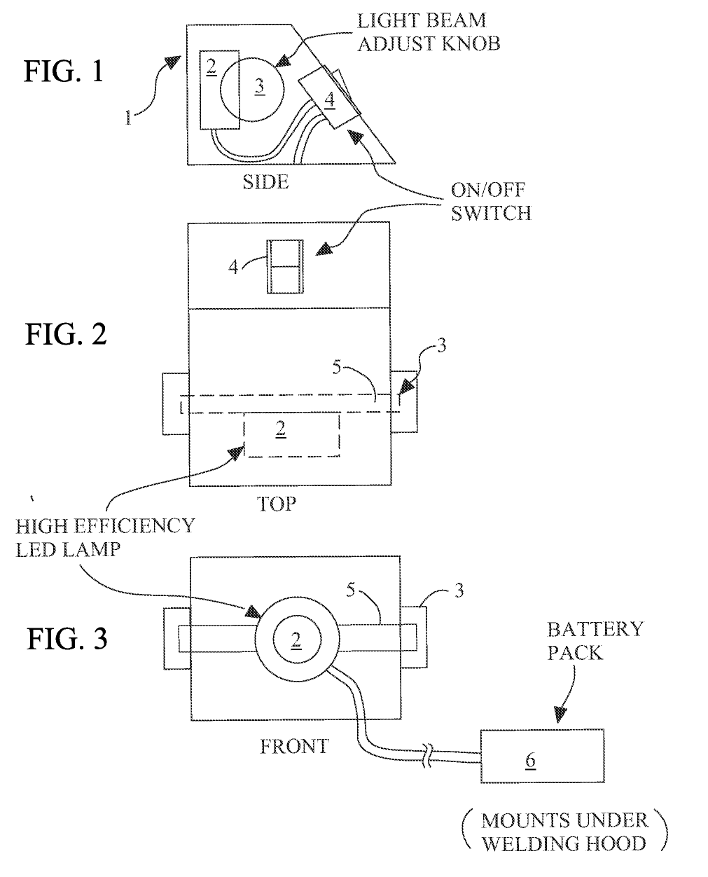

Referring to FIG. 1-3, there is disclosed a preferred embodiment of a light assembly 1 contained within a plastic housing.

FIG. 1 is a cross sectional view from the side of the light assembly 1 wherein a LED lamp 2 is contained in the front chamber of a plastic housing, a light beam adjust knob 3 is connected to the LED lamp 2, and the on/off switch 4 is connected to the LED lamp 2 by electronic circuit.

FIG. 2 is a cross sectional view from the top of the light assembly 1 wherein light beam adjustment knobs 3 are located on the side walls of the housing. The LED lamp 2 is attached to a rod 5 that transverses through the light assembly 1, reaches to both side walls of the housing and connects with light beam adjustment knob 3.

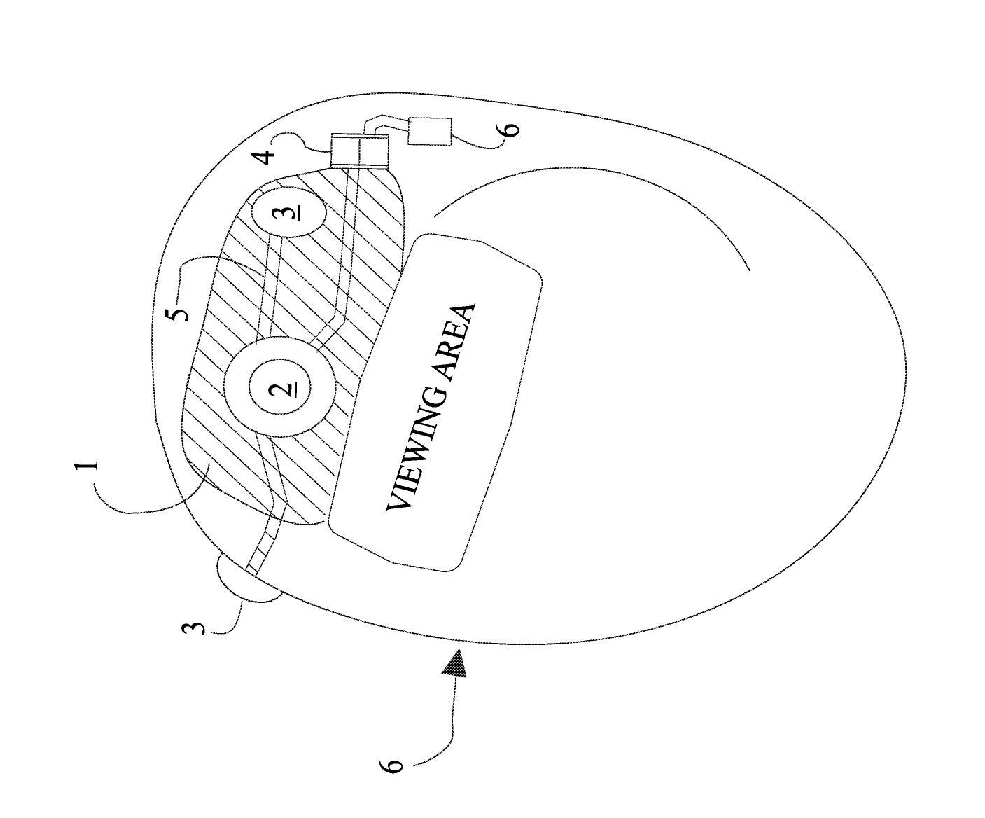

FIG. 3 is a cross sectional view from the front of the light assembly 1 wherein the LED lamp 2 is connected with the battery pack 6 through electronic circuits and on/off switch (not shown). The battery pack is mounted under welding hood.

FIG. 4 is a perspective view showing the light assembly 1 mounted onto the top of a welder helmet 6.

The housing has a generally right angled trapezium configuration. The housing is defined by a top wall, a bottom wall, a front wall, a back wall, opposed side walls, and a hollow chamber. The light source, high efficiency LED lamp 2 includes a plurality of LED disposed within the front part of the chamber. The LED lamp 2 may be covered with a clear plate. The adjustment knobs 3 may be included on the housing, which allow the user to position the light beam in the desired direction. In one of embodiments, the LED lamp 2 may be mounted to a rod 5 that traverses through the housing, reaches to both side walls of the housing and connects with rotatable knobs 3. Consequently, rotating the knobs 3, rotate the connected LED lamp 2. After each rotation, the LED lamp 2 will stay in place through a ratcheting mechanism thus the light beam can be positioned in the desired direction. FIG. 2 illustrates a cross sectional view from the top of the light assembly 1. This figure shows the position of light beam adjustment knobs 3 relative to LED lamp 2 and shows how the control knobs allow for adjustment of the lighting direction.

An on/off power switch 4 is disposed within the rear chamber of the housing. The on/off power switch is in communication with the LED lamp 2 and the power source 5 to control the flow of electrical energy from the power source 5 to the LED lamp 2. The on/off switch 4 extends outwardly to the back wall of the housing in FIG. 1. The on/off switch 4 may extends outwardly to the side wall of the housing in another embodiment. The power source 6 may be battery pack. The battery pack can be mounted for example through a clip-on- or snap-in connection, or with the help of slideable locking elements, that can be engaged and disengaged again by hand.

In general, LEDs vary in size, color output, and manufacturer. The LED selected for the preferred embodiment provides a broad spectrum so as to appear white to human eyes. The LED lamp 2 may further include a means to focus the light such as a lens to focus and change the light beam size. The high efficiency LED lamp 2 used in the embodiment is approximately 2″ in height, 3″ in length, and 2″ in width. It may be incorporated into the helmet during manufacture. The exact specifications may vary.

The advantage of using LED lamp for lighting is well known and does not need detail explanation here. White-light light emitting diode lamps have the characteristics of long life expectancy and relatively low energy consumption. The LED sources are compact, which gives flexibility in designing lighting fixtures and good control over the distribution of light with small reflectors or lenses. Due to the small size of LEDs, control of the spatial distribution of illumination is extremely flexible, and the light output and spatial distribution of a LED array can be controlled without efficiency loss.

The light assembly 1 may be mounted onto the top and/or face of a welding helmet 6. It may be mounted in such a way that it adjustably illuminates the area in front of the helmet's viewing window. FIG. 4 disclosed an embodiment of a welder helmet 6 wherein the light assembly 1 is incorporated onto the top of the welder helmet 6 during manufacture. The on/off power switch button 4 is located on the side of the helmet of this embodiment. The light direction adjustment knobs 3 are located on the side wall of the welder's helmet 6, which allow the user to easily access the knobs. By turning the knobs 3, the users can position the light beams in the desired direction. The light assembly 1 may also include a light intensity tuning function and include a tuning knob on the side of the welder helmet.

The light assembly 1 created as an independent unit is preferably mounted on the top or face of the welder helmet 6. It can be mounted for example through a clip-on- or snap-in connection, or with the help of slideable locking elements, that can be engaged and disengaged again by hand.

The foregoing description of specific embodiment of the present invention has been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. An adjustable light assembly for use on a welder helmet comprising:
a) a light emitting diode (LED) lamp;
b) an adjustment knob indirectly connected to the LED lamp to allow the user to position the light beam;
c) a power switch connected to the LED lamp;
d) a power source for the LED lamp coupled through the power switch;
e) an electrical connecting means disposed on the light assembly and helmet for electrically connecting the LED lamp, the power source, and the power switch; and
f) a connection means for attaching the light assembly to the welder helmet.

2. A welding helmet with adjustable LED assembly for illuminating a work area, wherein the adjustable light assembly comprising:
a) a light emitting diode (LED) lamp;
b) an adjustment knob indirectly connected to the LED lamp to allow the user to position the light beam;
c) a power switch connected to the LED lamp;
d) a power source for the LED lamp coupled through the power switch;
e) an electrical connecting means disposed on the light assembly and helmet for electrically connecting the LED lamp, the power source, and the power switch; and
f) a connection means for attaching the light assembly to the welder helmet.

3. The adjustable light assembly of claim 1 or 2 wherein the connection means for attaching light assembly to the welder helmet may include but is not limited to clip, and snap.

4. The adjustable light assembly of claim 1 or 2 wherein the LED lamp provides a bright white light source.

5. The adjustable light assembly of claim 1 or 2, wherein the power source comprising battery pack mounted under welding hood.

6. The adjustable light assembly of claim 1 or 2, wherein the electrical connecting means comprising electrical wire.

7. The adjustable light assembly of claim 1 or 2, wherein the LED lamp may be approximately 2 inch in height, 3 inch in length, and 2 inch in width.

8. The adjustable light assembly of claim 1 or 2 may be located onto the top or face of a welding helmet.

9. The adjustable light assembly of claim 1 or 2 further comprising a means for adjusting the light intensity.

10. The adjustable light assembly of claim 1 or 2 wherein the LED lamp further comprising means to focus the light.
